(19) United States
(12) Patent Application Publication
(10) Pub. No.: US 2005/0234477 A1
(43) Pub. Date: Oct. 20, 2005

(54) Method and apparatus for treating skin

(76) Inventors: Eva Matilda Brown, Paradise Waters (AU); William L. Mobbs, Farrar (AU)

Correspondence Address: WALLENSTEIN WAGNER & ROCKEY, LTD, 311 SOUTH WACKER DRIVE, 53RD FLOOR, CHICAGO, IL, 60606, US

(21) Appl. No.: 10/515,445

(22) PCT Filed: May 22, 2003
(86) PCT No.: PCT/AU03/00630

§ 371 Date: Jun. 13, 2005

(30) Foreign Application Priority Data
May 22, 2002 (AU) PS 2458
Aug. 19, 2002 (AU) 2002950849

(51) Int. Cl. A61B 17/50

(52) U.S. Cl. 606/131

(57) ABSTRACT

Methods and apparatus for removing skin blemishes and wrinkles by abrasion using a burr are disclosed. In one example a hand-held device (10) is used. It has a body (11) into the proximal end of which a low-voltage motor (14) is removably clipped and onto the distal end of which a shield or fitment (12) is removably attached. A rotary burr (20) is located within the shield so as to be rotatably coupled to the motor shaft. Preferably, the burr (20) and the shield (12) are attachable and removable as a single-use disposable fitment. The shield (12) may be located off-center with respect to the burr (20) so that rotation of the shield with respect to the body (11) will to expose more or less of the burr. A slot (32) may be formed in the wall of the shield (12) to discharge skin debris onto the skin being treated where it can be wiped away.

### TECHNICAL FIELD

This invention relates to methods and apparatus for removing skin blemishes, wrinkles and the like using a rotating abrasive burr.

The methods and devices of the invention are suitable for the reduction or removal of small brown skin blotches—sometimes called ‘liver spots’ or ‘age patches’—that appear on the face, chest and the backs of hands and, while related to sun exposure, are not freckles. The methods and devices are also suited to the reduction or removal of larger patches of brown skin discoloration, which appear on the faces and necks of women during pregnancy or soon after. In addition, the methods and devices of the invention are applicable to the reduction or removal of facial wrinkles and lines, particularly, though not exclusively, those in the perioral and periorbital areas. While the ‘depth of cut’ needed to remove liver spots is such that, with care, the procedure can be performed in a beauty salon, that required to remove most wrinkles and freckles is such that the procedure should be conducted by a doctor in surgery. In either case, however, the treated area is likely to ooze blood and/or serum during and after the procedure.

Accordingly, the method and apparatus of the invention can be distinguished from the grinding or sanding of calluses in podiatry where only dry, dead or heavily keratinized skin is removed. Also distinguished are strictly surgical procedures for burn-scar removal and bone or cartilage reduction.

### BACKGROUND TO THE INVENTION

The use of rotating wire-brushes or abrasive grinding wheels, motor-driven sanding discs and abrasive fingers, to remove skin blemishes has a long history, but is seldom seen in beauty salons or doctor's surgeries. The reasons seem to be, first, that these methods are generally not safe for the patient or client because they can easily result in the removal of too much skin and, second, that they are not safe for the practitioner because they tend to spray skin tissue about.

U.S. Pat. No. 2,867,214 to Wilson discloses the use of a hand-held rotary wire brush to remove facial blemishes while a refrigerant is sprayed on to the area being treated. A small arcuate shield covers a small portion of the brush to somewhat reduce tissue spray, but significant tissue spray will still occur and there is serious danger of removing too much skin, particularly if the color or texture of the skin is changed by the refrigerant. While motor-driven sanding drums used for podiatry [eg, that disclosed in French patent 2,728,777] also spread skin debris widely, the debris is likely to be dry (as already noted). In prior international patent application WO 01/13775, one of the present inventors disclosed the use of a small motor-driven rotary burr without any shield to remove liver spots but the problems of the spray of tissue spray and depth-of-cut control remained.

U.S. Pat. No. 6,391,034 discloses a surgical device for removing unwanted scar tissue from large burned areas, which is unsuitable for cosmetic purposes and employs a rotor comprising a wire brush, grinding cylinder or bladed cutter. The rotor is enclosed in a housing that is pressed against the scar tissue and aspirated—and if desired irrigated—to remove the large amount of tissue debris generated. The housing has adjustable sides by which more or less of the rotor can be exposed so that the depth of cut is thereby adjustable. Not only are the housing and associated plumbing bulky, but the use of this device also requires expensive associated aspiration and irrigation pumps along with careful disassembly and autoclaving after each use. It is therefore unsuitable for cosmetic purposes.

There are also various prior-art sanding devices. For example, U.S. Pat. No. 4,572,187 uses a motor driven rotor consisting of a hub with flaps of sandpaper extending from it. Again, excessive skin removal is a danger if used for cosmetic purposes and tissue debris is likely to be thrown over considerable distances, not to mention the danger of torn or shed flaps. Though spread of debris is lessened by the use of vibrating pads and fingers, as disclosed for example in U.S. Pat. Nos. 3,169,536 and 6,139,536 and in German patent 33 11 193, such abraders quickly clog up and become ineffective when removing liver spots, hormonal discoloration or wrinkles. Also, there is no provision for controlling the spread of tissue debris.

Finally, the prior art discloses means for near-total suppression of the scatter of skin debris by enclosing sanding pads [U.S. Pat. No. 6,387,103], router bits [U.S. Pat. No. 3,526,219] and burrs [U.S. Pat. No. 6,391,034 mentioned above] in shrouds or casings that are pressed against the skin and through which flushing liquid is pumped. Such systems are complex and expensive, difficult to keep sterile and require dismantling for autoclaving. They are only suited for use in hospitals where trained maintenance staff are available.

### OUTLINE OF THE INVENTION

From one aspect, this invention comprises a method of removing or reducing superficial skin discolorations or wrinkles involving attaching a disposable rotary burr and shield to a hand-held drive assembly, abrading the area of skin concerned while collecting at least portion of the skin debris generated in the shield, and then removing and disposing of the burr and shield. The method may include inhibition of egress of debris from the proximal end of the shield. Preferably, the method includes attachment and removal of the burr and shield as a unitary fitment, and may include the fitting of a thin flexible shroud, which forms part of the fitment, over the drive assembly while the fitment is being attached. It may also include the step of inverting the shroud over the shield prior to removal of the fitment.

The method may include the use of the shield to control depth of cut by contacting the shield with the skin and preferably discharging skin debris not caught in the shield through a slot in the shield onto the skin in the area of treatment so that it can be wiped away. From another point of view, the method of the invention may include use of the device as above indicated but wherein a motor driving the burr is controlled using a proximity switch so that the device does not need to be put down, and nor does the switch need to be touched, to effect control of the motor.

From another aspect, the apparatus of the invention may comprise a hand-held driver assembly that includes a rotary shaft located within a body. A burr is rotatably and detachably coupled to the shaft and a shield, which at least partially contains or surrounds the burr, is detachably attached to the body, the arrangement being such that portion of the skin debris is retained within the shield and can be disposed of with the shield.

Portion of the burr is preferably exposed through an opening in the shield so that the shield not only acts as a catcher for skin debris but also serves to limit contact of the burr with the skin, thereby providing a ‘depth of cut’ limit. A second opening or slot may be formed in the shield in such a way that skin debris not caught in the shield is discharged onto the skin in the vicinity of the shield.

The shield may be of tubular form having an open proximal end that is adapted to removably connect to or fit on the distal end of the body. Preferably, the shield is arranged so that it can be rotated relative to the body in such a manner as to expose more or less of the burr from the shield, thus varying the depth of cut.

The shield and burr may be arranged so that removal of the shield will also effect removal of the burr so that they may be fitted and disposed of together as a single fitment. It is envisaged that a thin flexible shroud may be attached to the shield so that it forms part of the fitment, the shroud serving to cover or protect at least portion of the driver assembly. Preferably, the burr has a stub-axle that extends from the proximal end of the shield, the stub axle and burr being rotatably located in the shield by bearing means and the stub-axle being adapted for coupling to the distal end of the shaft in the body. Sealing means may be provided as part of the fitment to inhibit movement of skin debris into the body of the driver assembly.

Accordingly, from another aspect, the invention may comprise a disposable fitment of the type indicated above as a single-use disposable manufactured product.

The shaft of the driver assembly may be rotated by a low-voltage motor housed within the body or by a flexible drive connected to a motor that is remote from the body or hand-piece. The use of a flexible drive that can be disconnected from the shaft allows the driver assembly to be sterilized or even autoclaved. Where the motor is housed within the body, it can be arranged for the motor to be removable so that the body alone, or preferably the body and shaft, can be sterilized or autoclaved. For example, the motor maybe slipped into place in the body from the proximal end thereof and held in position by clips.

The apparatus of the invention may be provided in two different forms, one suited to use by a medical practitioner in a clinic or surgery (where autoclaving is routine) and the other more adapted for use in beauty salons, where autoclaving is not generally available and operator expertise is less. The salon procedure will generally be used to remove liver spots and hormonal discoloration, which are superficial. Since the practitioner in the salon may not be as skilled as a doctor and lighting and other conditions may not match those of a surgery, the salon version will normally employ guards that limit depth of cut and minimize debris spread, even if the point of contact between the burr and the skin is thereby obscured. The use of disinfectant wipes to clean the body in the salon version will generally be satisfactory, given the protection provided by a fitment having a sheath.

As the clinical procedure will be generally used to remove or ameliorate perioral or periorbital wrinkles, deeper penetration of the skin will be required. Given the greater skill of the doctor and the superior conditions of a surgery, there is less need for the shield of the apparatus to limit the depth of cut. Moreover, many doctors prefer to see the point of contact between the burr and the skin as they work. Thus, in the clinical version, the opening in the shield may be larger than that for the salon version, or the control of depth of cut may be coarser. Also, given the availability of autoclaving, the clinical version may be one that uses a flexible drive that can be disconnected from the driver assembly so that this assembly can be autoclaved.

It will be appreciated that the present invention, as outlined above, offers significant benefits and improvements over and above those of the prior art.

### DESCRIPTION OF EXAMPLES

Having portrayed the nature of the present invention, a particular example will now be described with reference to the accompanying drawings. However, those skilled in the art will appreciate that many variations and modifications can be made to the example without departing from the scope of the invention as outlined above.

### BRIEF DESCRIPTION OF THE DRAWINGS

FIGS. 1A and 1B are respectively a side elevation a perspective view of apparatus comprising the first example.

FIG. 2 is an enlarged perspective view of the shield of the apparatus of FIG. 1, the shield being show removed from the remainder of the apparatus.

FIG. 3 is an enlarged side elevation of the shield fitted to the driver assembly of the apparatus of FIG. 1, the burr being partially visible.

FIG. 4 is an end elevation of the driver body, shield and burr in use.

FIG. 5 is an enlarged exploded perspective of portion of the distal end of the bottom of the body, shield and burr of a variant of the first example.

FIGS. 5A to 5D are various end elevations of the variant of FIG. 5, FIG. 5A being a view of the distal end of the body taken on plane VI-VI of FIG. 5, FIG. 5B being a view of the proximal end of the shield (without the burr in place) taken on plane VII-VII of FIG. 5, FIG. 5C being a view of proximal end of the shield with the burr in place when the shield is adjusted for a large depth-of-cut, and FIG. 5D being the same view as FIG. 5C with the shield adjusted for a small depth-of-cut.

FIGS. 6A to 6D are various views of a control unit for use with the device of the first example; FIG. 6A being a plan view of the control unit, FIG. 6B being a perspective view showing the transmitted and returned light beams, FIG. 6C is an elevation view of the control unit near a practitioner, and FIG. 6D is an elevation view of the control unit being operated by hand.

FIG. 7 is a perspective view of the device of the second example, shown fitted with a disposable fitment, partially disassembled and connected to a control unit of the type shown in the various parts of FIG. 6.

FIG. 8 is an exploded view of the disposable fitment shown in FIG. 7.

FIG. 9 is an enlarged sectional elevation of the distal portion of the fitment of FIG. 8.

FIG. 10 is a perspective view of the device of the third example wherein a flexible drive shaft is connected to the driver assembly.

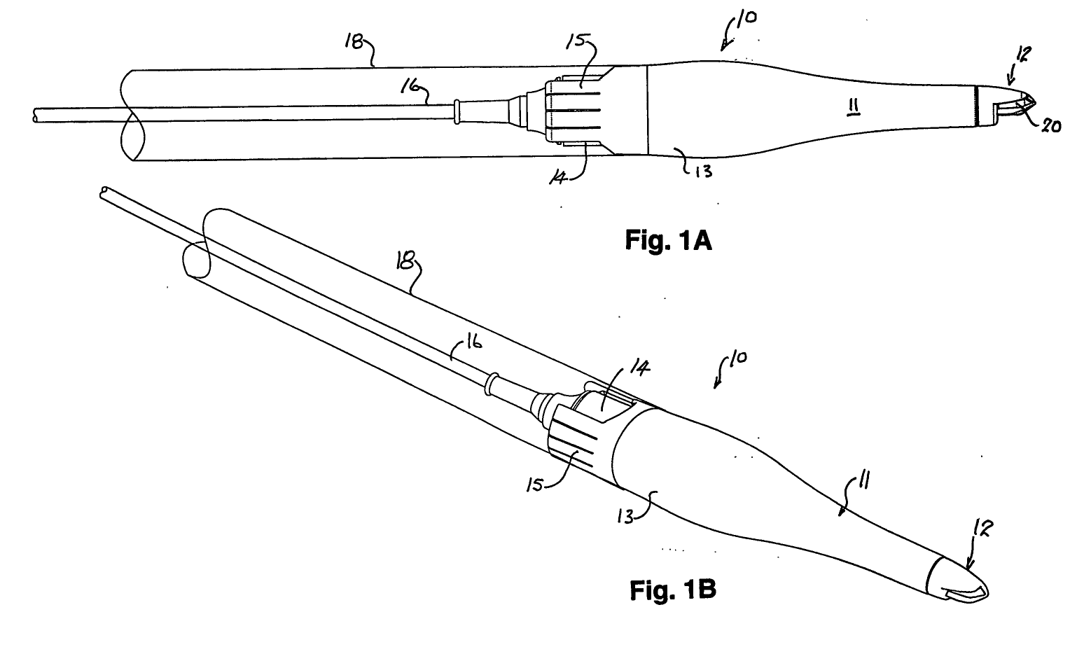

FIGS. 1A and 1B are two views of the device 10 of the first example, which comprises a driver assembly or hand-piece 11 fitted with a disposable shield 12 on its distal end, driver assembly 11 having a hollow tubular body 13 into the proximal end of which a high-speed low-voltage motor 14 and shaft (not shown) are inserted and held by clips 15, the power lead for motor 14 being shown at 16. In this example, a thin transparent tubular sheath 18 is fitted over clips 15 and the proximal end of driver assembly 11 to keep motor 14 and near portion of lead 16 clean. A burr 20, just visible in FIG. 1A, is fitted on to the end of the motor shaft (not shown) and is largely covered by shield 12.

We have found molded tubular abrasive paper or fabric burrs having a relatively coarse grit size—between 120 and 350—a diameter of a few millimeters and rotated at about 10,000 rpm are suitable. Abrasive elements of this nature are commercially available are formed from paper or fabric base and are generally cylindrical with integrally formed rounded or conical ends. They can be fitted onto an elastometric or plastic mandrel. Solid bonded burrs are also available and may be used. The small diameter of the burr allows the operator an excellent view of the portion of skin being abraded and facilitates the accurate location of the burr for ‘touch-up’ purposes. A tapered or rounded burr end allows treatment of small areas and fine wrinkles.

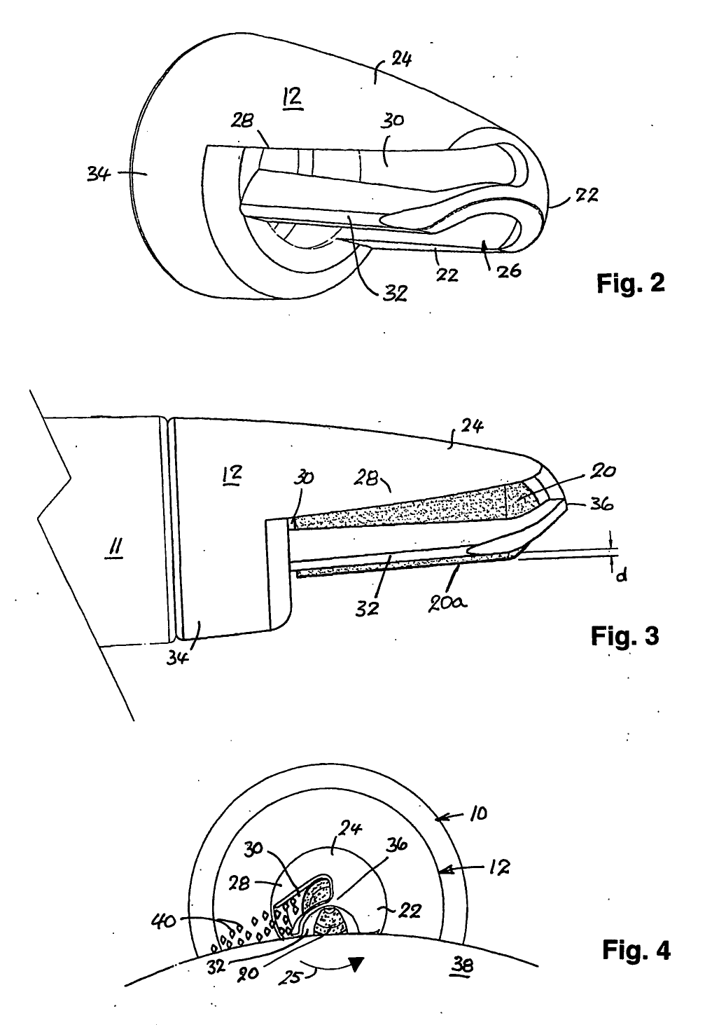

Referring to FIGS. 2-4, it will be seen that shield 12 is of a hollow conical shape having a rear or trailing sidewall 22 and a contiguous top wall 24 that follow the contour of the burr 20. [Note that burr 20—not shown in FIG. 2—is assumed to rotate in an anticlockwise direction as shown by arrow 25 in FIG. 4.] The bottom portion of the shield forms a longitudinally extending opening 26 through which the bottom part 20a of burr 20 is exposed. The leading or front sidewall 28 of the shield is cut away to form a slot 30 defined at the top by sidewall 28 and at the bottom by a bar 32 that extends from the circular proximal portion 34 of the shield to the distal end or nose 36 of shield 12 to meet rear sidewall 22. It will be seen that burr 20 extends below bar 32 and trailing edge 22 by a distance d, which determines the depth of cut.

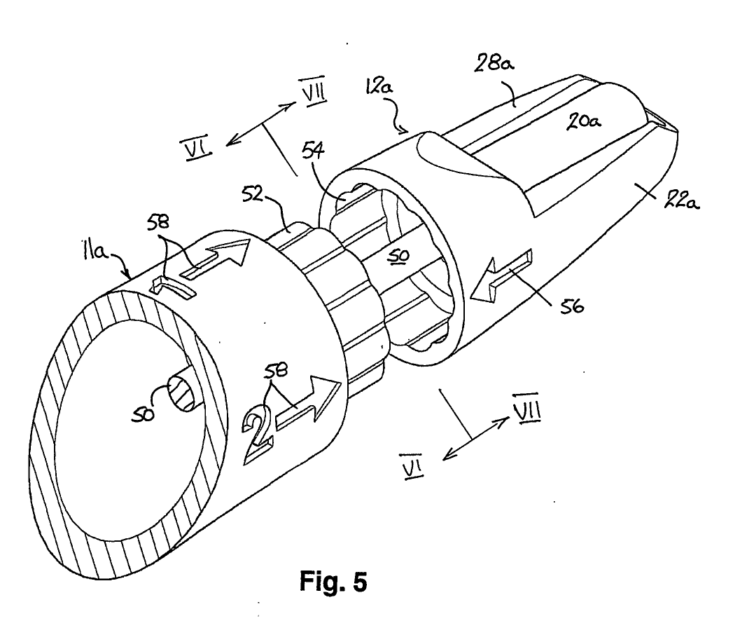

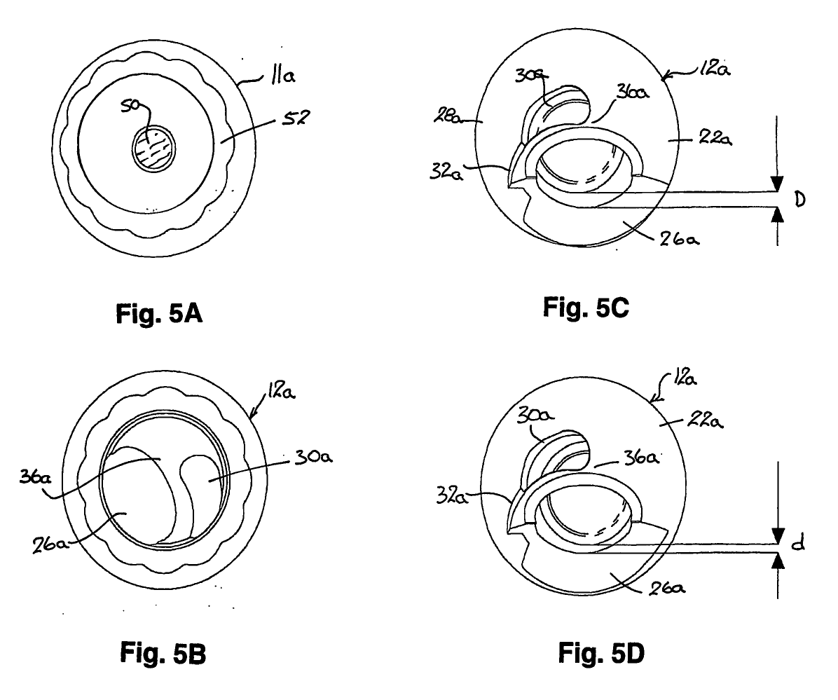

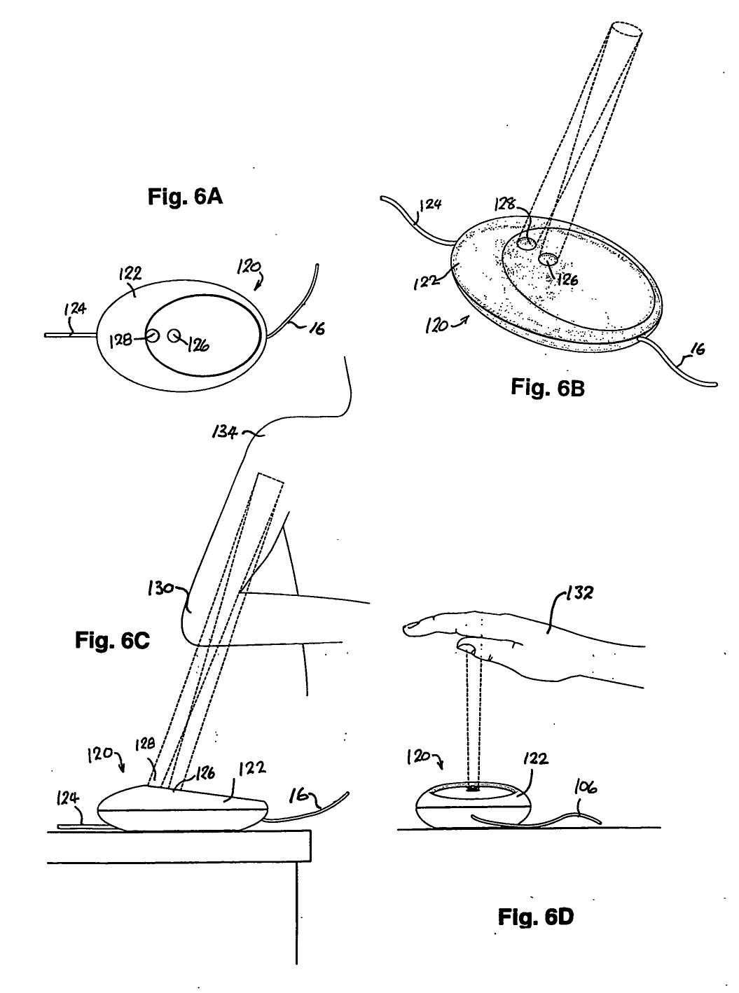

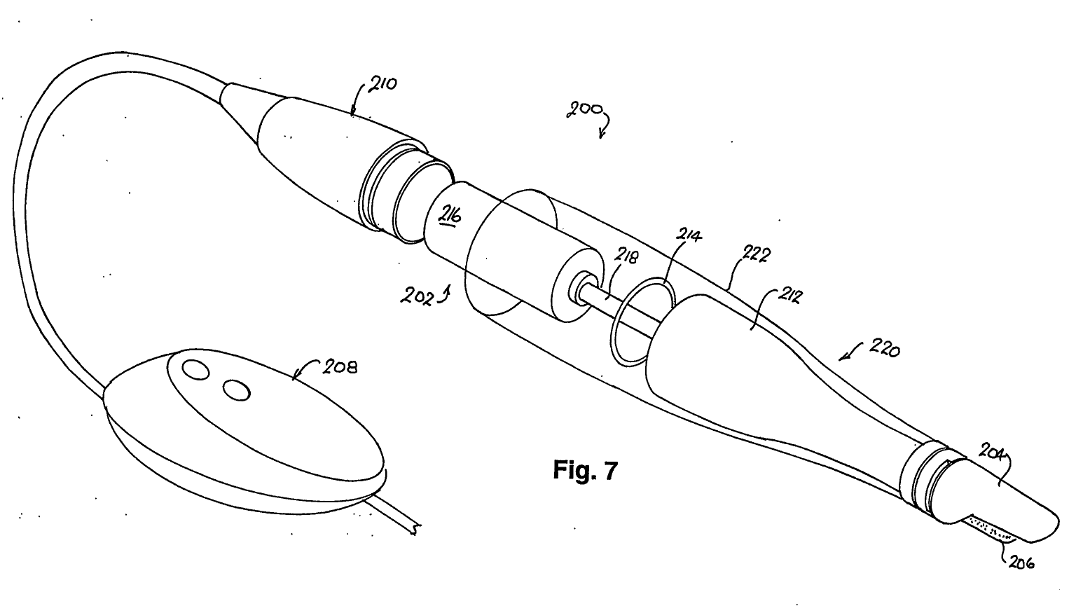

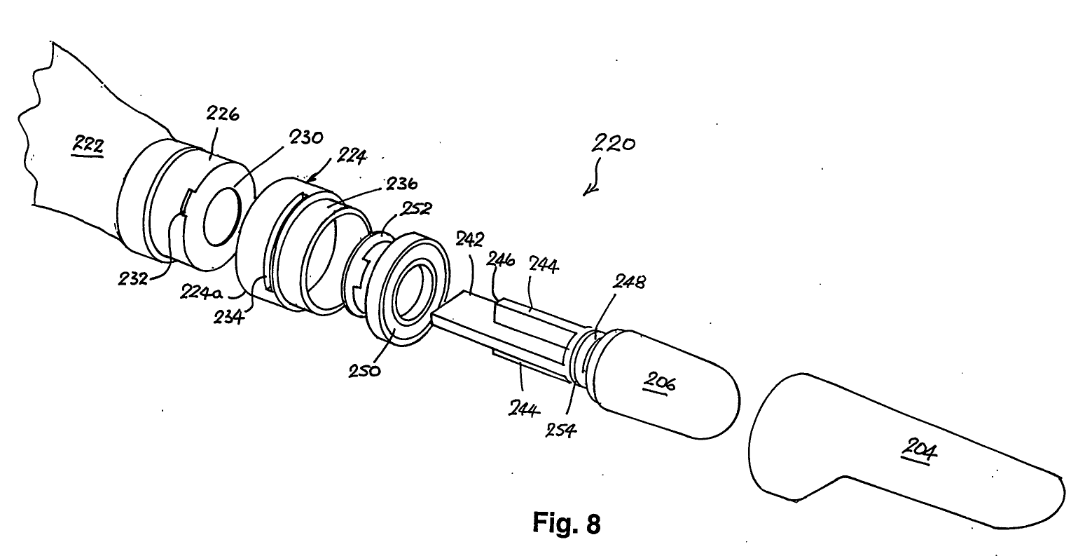

Referring particularly to FIG. 4, which is an end elevation of burr 20 and shield 12 in use, with the skin being abraded indicated at 38, it will be seen that bottom of bar 32 and trailing sidewall 22 rest on the skin 38 of the patient and thereby determine the amount of contact between then burr and the skin; that is, the depth of cut. While the amount of contact can be controlled by the operator to some degree by pressing shield 12 to the skin with greater or less force, coarse control of this variable can be provided by the use of burrs of different diameters or by substituting shields that expose more or less of the burr from the bottom thereof. However, a preferred method of providing adjustable control will be described below with reference to FIGS. 5 and 6A-6D.

Returning to FIG. 4, the manner in which most of the dead skin or debris 40 that is not retained in the shield is discharged from shield 12 onto skin 38 is illustrated. This occurs primarily through the slot 30 by virtue of air-entrainment by the burr, with air being drawn through the distal end of slot 30 in nose 36 of shield 12. Discharge of debris 40 may be assisted by blowing air into the proximal end of shield 12 through vents (not shown) in the front of motor 14 in driver 11. This will also inhibit ingress of debris into the distal end of driver body 11.

In practice, it is generally convenient for the practitioner to work by holding device 10 in the right hand so that slot 32 is visible and the burr is over the area of skin—say a liver spot—to be treated. The burr 20 is then brought into contact with the liver spot and moved away from the practitioner to expose the treated area, which is immediately wiped with a sterile or antiseptic tissue or swab so that the treated area can be clearly visualized. In this way, the removal of the darkened patch that forms the liver spot can be closely monitored and excessive tissue removal avoided. Since the area to be treated will have been thoroughly wiped with a moist antiseptic swab before treatment commences, it will be a little moist. This will result in most of the debris being retained in shield. However, some will be discharged through slot 32 onto the treated area of skin, where it will be wiped up and removed.

FIGS. 5 and 5A-5D illustrate a simple way in which different depths of cut can be set with a device that is essentially the same as device 10 described above. In describing this variant, the same reference numerals will be used as in the case of the device of FIGS. 1-4, except that the suffix ‘a’ will be applied. FIG. 5 is an enlarged and exploded view of the distal end of body 11a, shield 12a, burr 20a and the motor shaft 50, on which burr 20a is mounted, taken from the underside to more clearly show burr 20a. A scalloped boss 52 of reduced diameter forms the distal end of body 11a and a complimentary scalloped bore 54 is formed in the proximal end of shield 12a to provide a firm push fit onto boss 52. Bore 54 is concentric with shield 12a, while boss 52 is eccentric with respect to the axis of shaft 50 and burr 20a. Thus, the depth of cut can be set by setting the relative angular alignment of body 11a and shield 12a, which may be done when the shield is pushed into place on body 11a or by rotating the shield in a clicking or detenting manner after it is has been pushed onto the body. An arrow 56 on shield 12a provides a witness mark, while numerals and arrows 58 on the body indicate the cut-depth setting.

The means by which variation of cut-depth is achieved is illustrated in more detail in FIGS. 5A-5D, FIG. 5A being an end elevation of the distal end of body 11a taken from plane VI-VI of FIG. 5 with the shield 12a and burr 20a removed, and FIG. 5B being an end elevation of the proximal end of shield 12a taken from plane VII-VII with shaft 50 and burr 20a removed. The eccentricity of boss 52 with respect to shaft 50 and the concentricity of bore 54 with respect to shield 12a has been exaggerated for clarity. FIG. 5C is an end elevation of shield 12 with burr 20 in place (but with body 11a omitted for clarity), the angular alignment of the shield with respect to the body being set to provide the maximum depth of cut D. In FIG. 5D, the angular alignment of the shield 12a and the body (also omitted in this Figure) is such as to provide the minimum depth of cut d. Normally the difference in depth is less than a tenth of a millimeter.

In order to allow motor 14 of device 10 to be switched on and off without the need to put down the device or touch a switch, switching means comprising a control unit 120, illustrated in FIGS. 6A to 6D, is used in the first example. Control unit 120 has a flat lozenge shape body 122 that has an input power lead 124 and output motor lead 16 [see also FIGS. 1A and 1B]. A pulse-coded infrared transmitting LED 126 is located in body 122 along-side an infra-red sensor 128 connected to a receiver circuit (not shown) within body 122 that is tuned to the coding of the LED 126. LED 126 and sensor 128 are fitted with lenses such that the positioning of a near object—such as an elbow 130 in FIG. 6C or a hand 132 in FIG. 6D—will reflect sufficient of the transmitted beam to be detected by sensor 128 and its receiver. This can be made to turn the power to the hand-piece 11 off. However, the transmitting LED 126 and the sensor 128 are arranged so that the interruption of the beam by a more distant object—such as the shoulder 134 of the operator 134 in FIG. 6C—will not be detected.

It is envisaged that the control circuit (not shown) in control unit 120 may be wired so that the power to the hand piece can be varied. For example, successive interruptions to the beam can cycle the control through two or three power settings as well as off/on. Control circuits suitable for use in controller 120 are known in the art and appropriate chip sets are available from a variety of manufacturers.

The remote operation of the hand piece control is of great value in ensuring cleanliness. A foot switch and control unit may be used instead, but it has been found to be less convenient.

The second example of apparatus formed in accordance with the invention will now be described with reference to FIGS. 7-10.

The device 200 of the second example is shown in perspective in FIG. 7 with the hand-piece or driver assembly 202 partially disassembled but with a shield 204 in place over the burr 206. As it is assumed that device 200 is for clinical use for the removal of facial wrinkles, no side slot is provided in the shield so that much more of burr 206 is exposed than in the first example. This enables the doctor to see the point of contact between the burr and the skin, but relies upon his/her skill to regulate the depth of cut. As in the first example, power to the driver assembly is of low voltage and toggled on and off using a light-beam switch 208 as described with respect to the various parts of FIG. 6.

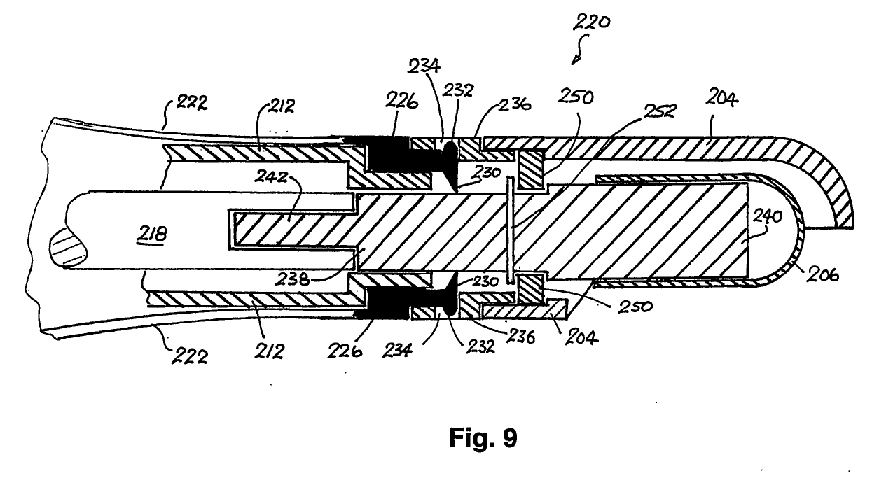

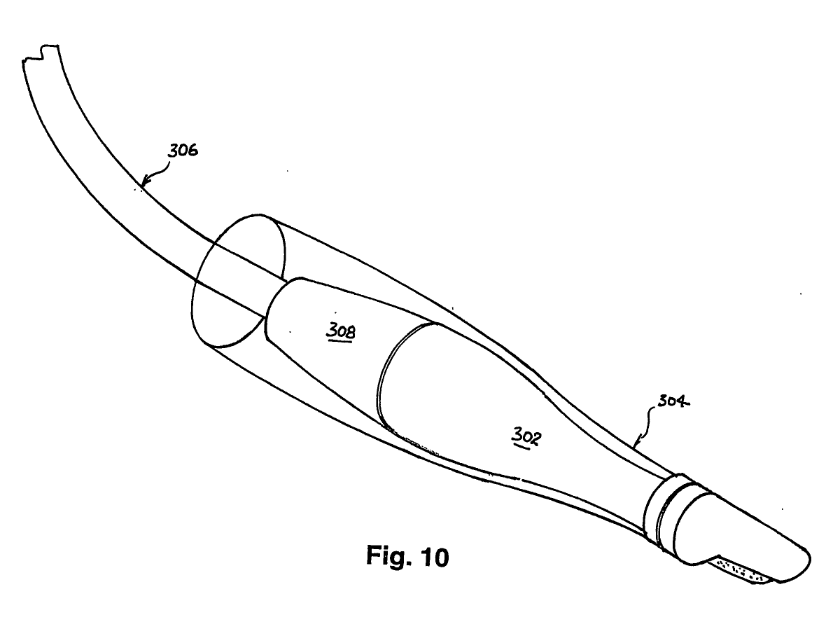

Driver assembly 202 is split transversely into a rear cap 210 and a front tubular portion 212, which can be screwed or snapped together with O-ring 214 to enclose a high-speed low-voltage motor 216 and its shaft 218 that can be direct-coupled to burr 206. In this example, shield 204 is part of a more complex disposable single-use fitment 220 that is attached to the distal end of front portion 212 of driver 202. In addition to shield 204 and burr 206, fitment 220 includes a thin flexible transparent plastic sheath 222 that is joined to the guard by an intermediate collar 224 and various internal parts associated with burr 206 that will be described with reference to FIGS. 8 and 9.

Referring, then, to FIGS. 8 and 9, sheath 222 has co-molded ferrule 226 (shown in black in the section drawing of FIG. 9), which fits onto a shoulder 228 (FIG. 9) formed on the distal end of the front portion 212 of driver assembly 202 and which has an inwardly extending blade-seal 230. Ferrule 226 is molded from a resilient plastic and has a pair of key-like tabs 232 (only one being visible in FIG. 8) extending radially from its distal face, which forms blade-seal 230. Intermediate collar 224 as a cylindrical proximal portion 224a that has a pair of circumferentially extending slots 234, and a distal cylindrical portion 236 of lesser diameter. Collar 224 is pressed onto ferrule 226, deflecting tabs 232 and distorting ferrule 226 until tabs 232 snap into slots 234, locking the two components together while permitting limited angular relative movement of tabs 232 in slots 234.

Burr 206 is a hollow cylinder having its proximal end open and its distal end closed and of semi-spherical or conical shape. It can be moulded of paper or fabric material that is impregnated with resin into which abrasive particles are set (as is well known in the abrasives art). Burr 206 is pressed onto a stub-axle 238, which has a distal cylindrical mandrel 240 onto which burr 206 is pressed and a proximal driving tongue 242 that fits in a slot formed in the distal end of motor shaft 218. Opposing splines 244 rise transversely from tongue 242 to provide a stop or shoulder 246 that abuts the end of shaft 218. A short bearing journal 248 is formed between the distal ends of splines 244 and mandrel 240 to take a small ball bearing 250, which is located axially by a circlip 252 that snaps into a groove 254 formed at the proximal end of journal 248.

The components of fitment 220 are assembled as follows: burr 206 is fitted onto mandrel 240, bearing 250 is pressed onto journal 248 and circlip 252 it clipped into groove 254 to hold bearing 250 in place. The proximal end of shield 204 is then fitted over distal end 236 of collar 224 and the two parts are bonded together before proximal portion 224a of collar 224 is snapped in place onto ferrule 226 of shroud 222, as previously described. The completed fitment 220 is then sterile packed ready for distribution and use. As will be obvious, fitment 220 is fitted onto the distal end of body 212 by slipping driver assembly 202 into the open end of shroud 222, manipulating fitment 220 and assembly 202 until tongue enters the slot in the distal end of shaft 218 and then fitting ferrule onto the spigot on the end of body 212.

The ability to rotate shield 208 relative to ferrule 226 can be used in two ways: to adjust the angle of shield to better suit the practitioner when using the clinical-type shield 204 or to vary the depth of cut (as previously described) when a salon-type shield (like that of the first example) is employed.

Finally, the third example shown in FIG. 10 comprises a device 300 that is a minor variant on the second example of FIGS. 7-9 in that, in place of a motor within the hand piece, a flexible drive may be used. Accordingly, as shown in FIG. 10, device 300 comprises essentially the same hand-piece body 320 and fitment 304 as the previous example, but a flexible drive shaft 306 is coupled onto body by a removable cap 308 so as to rotatably connect with the proximal end of the shaft (not shown) in body 320. This allows the body and shaft to be autoclaved if desired, once the flexible drive 306 and fitment 304 have been removed.

While examples of the invention have been described with reference to drawings, it will be appreciated by those skilled in the art that many other examples can be devised and many variations and modifications to the example disclosed can be made without departing from the invention as defined by the following claims.

1. A method of removing discolorations or wrinkles in an area of skin to be treated, comprising the steps of:
attaching a disposable rotary burr and a disposable non-rotary shield to a hand-held drive assembly so that the shield at least partially surrounds the burr,
rapidly rotating the burr using the drive assembly while moving the burr in contact with the area of skin to be treated so that a first portion of skin debris is retained in the shield,
removing the shield and burr from the drive assembly after the skin has been treated, and
disposing of the shield and burr.

2. A method according to claim 1 wherein the burr and shield comprise a disposable fitment for the drive assembly and the method includes the steps:
of attaching the fitment as a unit to the drive assembly, and removing and disposing of the fitment as a unit after use.

3. A method according to claim 2 wherein the fitment includes a thin flexible tubular shroud attached to the shield, the method including the steps of:
attaching the fitment to the drive assembly so that the shroud covers at least portion of the drive assembly, and after use
removing of the fitment, inclusive of the shroud, from the drive assembly as a unit for disposal.

4. A method according to claim 3 wherein removal of the fitment includes the steps of:
peeling the shroud off the driver assembly while causing it to invert over the shield and the burr; and
gripping the shield and burr through the inverted shroud to effect detachment of the fitment of the driver assembly.

5. A method according to claim 1 including the step of:
maintaining contact between the shield and the skin while moving the rotating burr in contact with the skin to thereby limit or control the depth of skin removed by the burr, and
returning a second portion of skin debris generated to the area of skin, and
wiping the area to remove said second portion of skin debris.

6. A method according to claim 1 wherein the driver assembly can be energized by switch means that transmits an IR beam and detects IR energy reflected from an object that intercept the beam, the method including the steps of:
locating the switch means so that the IR beam will be generally vertical and in the vicinity of the motor assembly during the treatment, and
controlling the drive assembly by elbow movement to intercept the beam without touching the switch means or putting the drive assembly down.

7. Apparatus for use in treating skin to reduce superficial skin blemishes or wrinkles, comprising:
a hand-piece including a driver assembly having a body with a proximal and distal end,
a rotatable shaft within said body having a proximal and a distal end, the distal end of the shaft being located near the distal end of the body,
a rotatable burr removably coupled to said distal end of said shaft for coaxial rotation thereby,
a shield covering enough of said burr to catch and retain a first portion of skin debris removed by the burr when the apparatus is in use, said shield having an open proximal end by which it is removably attached to the distal end of the body.

8. Apparatus according to claim 7 wherein the burr is located within the shield so that attachment of the shield to the distal end of the body effects the rotatable coupling of the burr to the shaft and so that detachment and removal of the shield effects the uncoupling of the burr from the drive shaft and the removal of the burr.

9. Apparatus according to claim 7 wherein:
said shield substantially encloses the burr, but has an opening through which portion of the burr protrudes so that it can be brought into contact with the skin to be treated, and
wherein the size or shape of said opening limits the surface area of the burr that can be brought into contact with the skin to be treated.

10. Apparatus according to claim 7 wherein:
the shield is rotatably attached to the body about an axis that is not coaxial with the burr, so that rotation of the shield with respect to the body adjusts the amount of burr protruding from the opening.

11. Apparatus according to claim 7 wherein a slot is formed in the shield so that a second portion of skin debris removed by the burr, when the apparatus is in use, will be discharged through said second opening onto skin external to the shield.

12. Apparatus according to claim 7 wherein;
the burr and shield form at least part of a disposable fitment that can be attached as a unit to the distal end of the body for use, and then detached as a unit from the distal end of the body for disposal after use.

13. Apparatus according to claim 12 wherein:
the burr is mounted on a stub-axle having a proximal end adapted for coupling to the distal end of the shaft, and
the stub-axle is rotatably mounted within a bearing that is located within and by the shield, so that rotatable coupling of the stub axle and burr to the shaft is effected by attaching the shield to the distal end of the body and so that detachment of the shield from the distal end of the body also effects uncoupling of the stub axle from the shaft.

14. Apparatus according to claim 12 wherein:
the stub axle extends through and from the proximal end of the shield, and
a seal is located between the stub axle and the shield lifted so as to inhibit the passage of skin debris from the proximal end of the shield.

15. Apparatus according to claim 12 wherein:
the fitment includes a thin flexible shroud that is attached to the proximal end of the shield and extends proximally from the shield so as to substantially cover the body of the hand-piece when the fitment is attached thereto, the shroud being removable from the body as part of the fitment.

16. Apparatus according to claim 15 wherein:
the shroud has a distal end that is formed as or attached to an intermediate collar,
said intermediate collar is joined to the shield, so that the shroud, intermediate collar and shield are adapted to be attached to and detached from the body as a unit, and
said intermediate collar includes an inwardly turned flange adapted to form a seal that inhibits the ingress of skin debris into the body of the hand piece.

17. Apparatus according to claim 16 wherein:
the shield is rotatable through a limited angle with respect to the intermediate collar so as to adjust the amount of burr protruding through the opening in the shield.

18. Apparatus according to claim 7 wherein:
the body is adapted to be held and manipulated in one hand to apply the burr to the skin,
the body includes a low-voltage electrically-powered motor arranged to rotatably drive the shaft;
a power lead is connected to the motor and extends from the proximal end of the body to a switch unit,
when the apparatus is in use, the switch unit is adapted to detect the interception of a beam of light by an object and to thereby effect on/off control of the motor.

19. Apparatus according to claim 7 wherein:
the body is adapted to be held and manipulated in one hand to apply the burr to the skin,
the proximal end of the shaft is adapted for rotary coupling to a flexible drive, and
the body and shaft are adapted for sterilization by autoclaving after removal of the flexible drive shaft and the burr and shield.

20. A disposable fitment for use with a hand-held driver assembly having a body with a distal end, the fitment comprising:
a tubular shield having an open proximal end adapted for connection to the distal end of the body, and
a rotary burr located within the shield.

21. A disposable fitment according to claim 20 and including:
bearing means within the shield,
a stub axle on the burr extending proximally from the proximal end of the shield, the stub axle being rotably supported by the bearing means within the shield, and
seal means arranged between the shield and the stub axle to inhibit egress of skin debris from the proximal end of the shield.

22. A disposable fitment according to claim 20 and including a thin flexible tubular shroud having a distal end connected to the proximal end of the shield, said shroud being adapted to cover the body when the proximal end of the shield is connected to the distal end of the body.
